(19) United States
(12) Patent Application Publication
(10) Pub. No.: US 2007/0015967 A1
(43) Pub. Date: Jan. 18, 2007

(54) Autosteering vision endoscope

(75) Inventors: Dennis R. Boulais, Danielson, CT (US); Michael S. Banik, Bolton, MA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Boston Scientific Scimed, Inc., Maple Grove, MN (US)

(21) Appl. No.: 11/524,629

(22) Filed: Sep. 20, 2006

Related U.S. Application Data
(63) Continuation of application No. 10/406,148, filed on Apr. 1, 2003, now abandoned.

(51) Int. Cl. A61B 1/00; A61B 1/04

(52) U.S. Cl. 600/146; 600/152; 600/118

(57) ABSTRACT

A control mechanism for automatically steering the distal end of an endoscope into a body cavity. During advancement of the endoscope, the control mechanism directs the distal tip to be moved in a sweep pattern. Images obtained during the sweep pattern are combined and analyzed in order to determine the desired direction of advancement of the endoscope. The distal end of the endoscope is then oriented in the direction of the desired advancement direction. An autoexamination feature can be activated that automatically moves the distal tip in a desired sweep pattern. Images of a body cavity are obtained during each sweep pattern for analysis by the physician.

### CROSS-REFERENCE TO RELATED APPLICATION

The present application is a continuation of U.S. patent application Ser. No. 10/406,148, filed Apr. 1, 2003, the benefit of which is claimed under 35 U.S.C. § 120 and is herein incorporated by reference.

### FIELD OF THE INVENTION

The present invention relates to medical devices in general and imaging endoscopes in particular.

### BACKGROUND OF THE INVENTION

As an aid to the early detection of disease, many doctors are recommending that patients undergo regular endoscopic examinations of such internal organs as the esophagus, lungs, colon, uterus, etc. A conventional imaging endoscope used for such procedures comprises an elongate tube with a fiber optic light guide that directs light from an external light source to a lens at the distal end of the endoscope. A gathering lens and fiber optic imaging light guide transmit an image to a camera or viewer at the distal end of the endoscope. In addition, most endoscopes include one or more working channels through which medical devices such as biopsy forceps, snares and other tools may be passed.

When an endoscope is used for performing a procedure such as a colonoscopy, a physician navigates the distal end of the endoscope to the cecum or junction of the small and large intestines. The pathway to the cecum is highly tortuous, often requiring considerable skill and strength of the physician in order to fully advance the endoscope. Most endoscopes have a manual control that moves the tip of the endoscope up and down as well as another manual control that moves the tip left and right. In practice, many physicians guide the scope using a single control while simultaneously twisting the endoscope in order to advance it in the desired direction. In some cases, the endoscope may become so twisted in the patient that it is difficult for the physician to use the viewing system of the endoscope to find the direction in which the endoscope should be advanced.

Once at the desired position, the physician performs an examination by withdrawing the scope while moving the distal end in a search pattern in order to view a body cavity as the scope is being withdrawn. If an area of tissue appears to be of interest, a surgical procedure can be performed before further withdrawing the scope. In some cases, the twisting of the scope makes it difficult for the physician to steer the distal end of the endoscope in the search pattern, and there is a chance that the physician may miss areas of the body cavity as the endoscope is being withdrawn.

Given these problems, there is a need for a mechanism for more easily inserting an endoscope into a tortuous body cavity and for performing an examination procedure that is less likely to miss areas of interest.

### SUMMARY OF THE INVENTION

The present invention is a control mechanism for steering an imaging endoscope of the type having a light source and an image sensor at its distal end and one or more actuators that operate to orient the distal tip in a desired direction. In accordance with one embodiment of the present invention, images obtained from the image sensor are combined to determine a desired direction of advancement within a patient. The actuators are then controlled to orient the distal tip in the desired direction.

In accordance with another aspect of the present invention, the control mechanism causes actuators to automatically move the distal tip of the endoscope in a search pattern as the endoscope is moved. A physician can then advance or retract the endoscope to view and/or record images created during the search pattern in order to perform an endoscopic examination.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates one embodiment of an endoscopic video imaging system with which the present invention may be used;

FIG. 2A is a block diagram of a motion control cabinet with which the present invention may be used;

FIG. 2B is a block diagram of another embodiment of a motion control cabinet with which the present invention may be used;

FIG. 3 illustrates one method of determining an advancement path in a body cavity in accordance with the present invention;

FIG. 4 illustrates a number of images obtained from the endoscope that are combined to allow determination of an advancement path in a body cavity in accordance with the present invention; and

FIG. 5 illustrates one method of operating the endoscope to automatically examine a body cavity in accordance with another aspect of the present invention.

### DETAILED DESCRIPTION OF THE PREFERRED EMBODIMENT

As discussed above, the present invention is a control mechanism for directing an endoscopic imaging system within a tortuous body cavity such as the colon. The control mechanism of the present invention also directs the distal tip of the endoscope in a search pattern in order to perform an examination of a body cavity. The present invention may be used with any actuator controlled endoscopic imaging system such as that described in U.S. patent application Ser. No. 10/406,149, which is herein incorporated by reference.

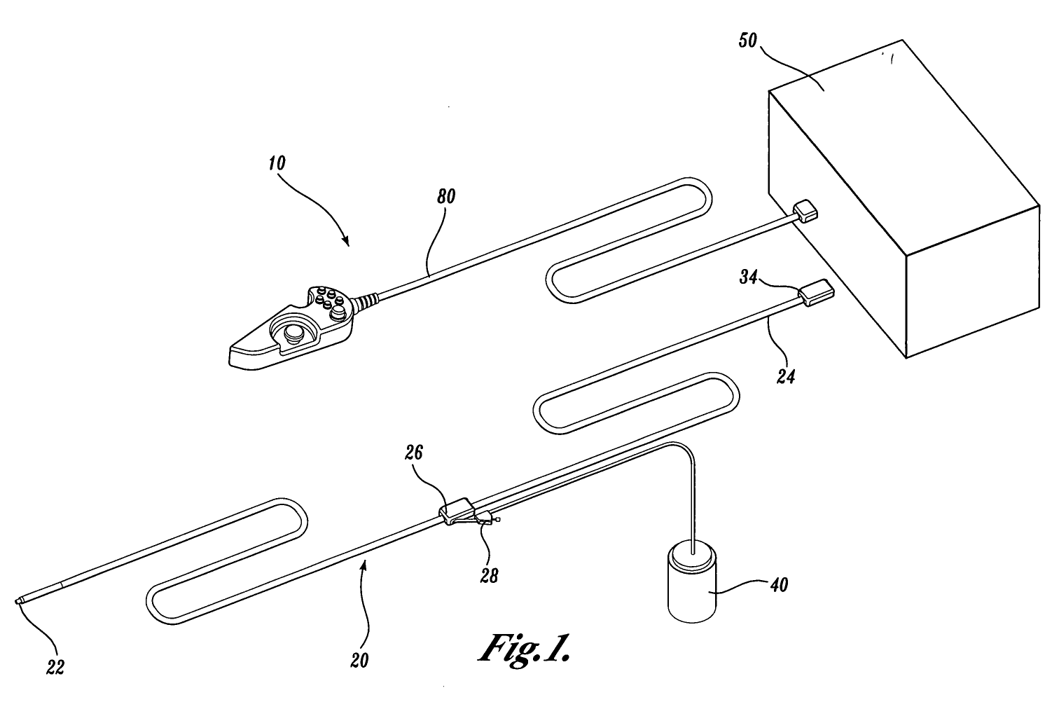

As shown in FIG. 1, an endoscopic video imaging system 10 with which the present invention can be used includes an imaging endoscope 20, a motion control cabinet 50 and a handheld controller 80. The imaging endoscope 20 has a distal tip 22 that is advanced into a patient's body cavity and a proximal end 24 that is connected to the motion control cabinet 50. The motion control cabinet 50 includes a number of actuators (not shown) that control a steering mechanism within the endoscope in order to change the orientation of the distal tip 22. A physician or their assistant uses the handheld controller 80 to input control signals that move the distal tip 22 of the imaging endoscope 20. In addition, the motion control cabinet 50 may include a connection to sources of air/gas and a flushing liquid such as water for clearing the imaging endoscope. The motion control cabinet 50 also includes imaging electronics to create and/or transfer images received from an image sensor to a video display for viewing by a physician or technician.

In the embodiment shown, the imaging endoscope 20 also includes a breakout box 26 that is positioned approximately midway along the length of the endoscope. The breakout box 26 provides an attachment point for a vacuum bottle 40 that collects liquids from a lumen within the imaging endoscope. The vacuum bottle 40 is controlled by a vacuum valve 28 that is positioned on the breakout box 26. Alternatively, the valve can be positioned within the motion control cabinet 50 and controlled from the handheld controller 80. The physician can insert surgical instruments such as biopsy forceps, snares, etc., into a working channel lumen of the endoscope in order to perform a surgical procedure at the distal end of the endoscope.

If desired, the handheld controller 80 can be secured to the breakout box 26 such that the two units can be moved as one if desired. Upon completion of a patient examination procedure, the imaging endoscope 20 is disconnected from the motion control cabinet 50 and thrown away. A new imaging endoscope 20 is then connected to the motion control cabinet 50 for the next examination procedure to be performed.

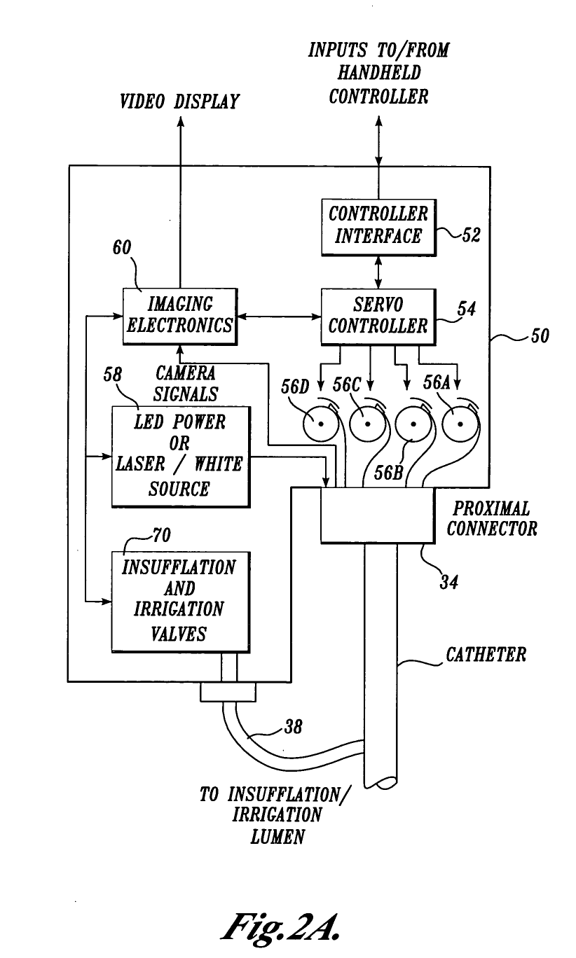

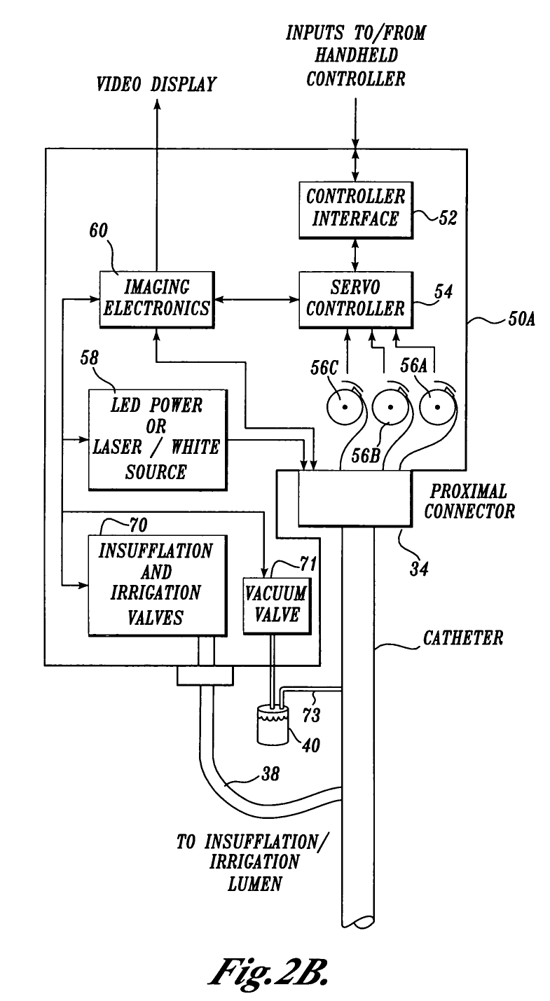

FIG. 2A is a block diagram of the major components included within one embodiment of the motion control cabinet 50. The motion control cabinet is preferably positioned on a cart that is wheeled near a patient prior to an examination procedure. The motion control cabinet is connected to a source of electrical power, either A.C. mains or a battery, as well as to a source of insufflation gas and irrigation liquid. Inside the motion control cabinet 50 is a controller interface 52 that is connected to the handheld controller 80 and receives control signals therefrom. To change the orientation of the distal tip of the imaging endoscope, the control signals are received from a directional switch in the handheld controller 80. The control signals are supplied to a servo motor controller 54 that in turn controls a number of actuators, such as servo motors 56a, 56b, 56c, 56d. Each of the servo motors 56a-56d is connected to one or more control wires within the imaging endoscope. Motion of the servo motors 56a-56d pulls or releases the control wires in order to change the orientation of the distal tip 22 of the imaging endoscope 20. Although the embodiment shown in FIG. 2A shows four servo motors and control wires, it will be appreciated that fewer or more servo motors and corresponding control wires could be used to move the distal tip. For example, some imaging endoscopes may use three control wires and three associated servo motors.

Also included in the motion control cabinet 50 is a power source 58 that provides electrical power to a light source such as a number of light emitting diodes (LEDs) at the distal end 22 of the imaging endoscope. Alternatively, if the imaging catheter utilizes an external light source, then the motion control cabinet can include a high intensity light source such as a laser or Xenon white light source that supplies light to a fiber optic illumination guide within the imaging endoscope 20 in order to illuminate an internal body cavity. The power source 58 may be controlled by control signals received from the handheld controller 80 when the user desires to activate the light source.

An imaging electronics board 60 and one or more microprocessors capture images received from an image sensor (not shown) at the distal end of the imaging endoscope. The imaging electronics board 60 can enhance the images received or can provide video effects such as zoom, color changes, highlighting, etc., prior to display of the images on a video display (not shown). Images produced by the imaging electronics board 60 may also be printed on a digital printer, saved to a computer readable media such as a floppy disk, CD, DVD, etc., or a video tape for later retrieval and analysis by a physician.

Finally, the motion control cabinet 50 includes valves 70 that control the delivery of insufflation air/gas to insufflate a patient's body cavity and an irrigation liquid to flush out a body cavity and/or clean the imaging light source and image sensor at the distal end of the endoscope. The insufflation air/gas and irrigation liquid are connected to the imaging catheter via a connector 38 that connects to an irrigation/insufflation lumen of the imaging endoscope 20.

FIG. 2B illustrates another embodiment of a motion control cabinet 50A that is similar to the cabinet shown in FIG. 2A. The motion control cabinet 50A includes a vacuum valve 71 that controls vacuum delivered to a vacuum collection bottle 40. A vacuum line 73 connects to a vacuum lumen within the imaging endoscope 20. The vacuum valve 71 is controlled from the handheld controller 80.

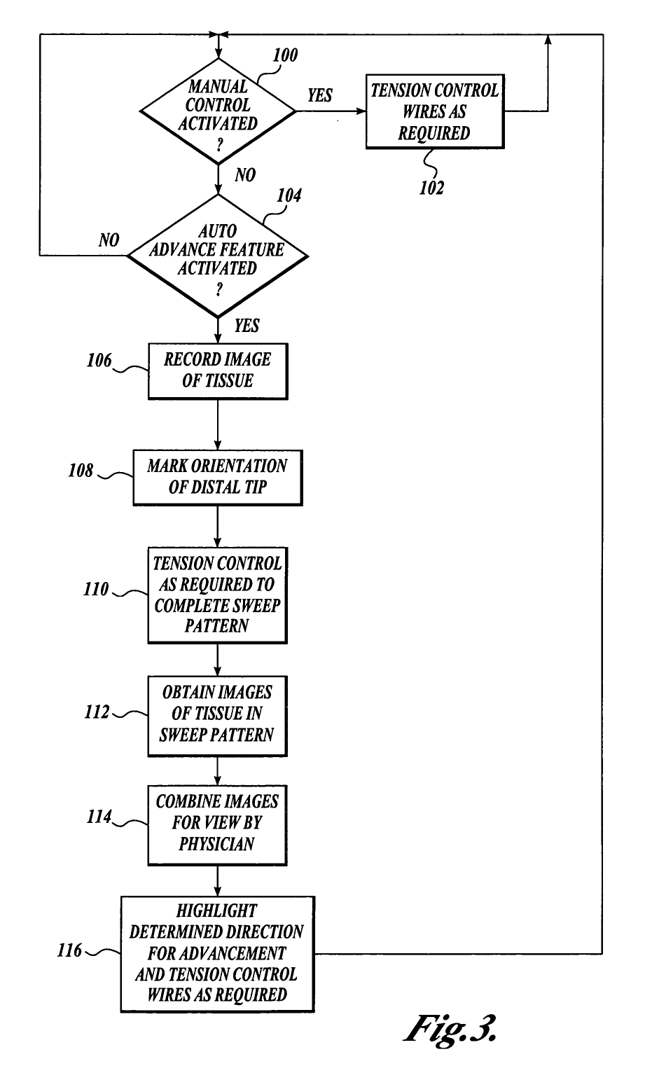

As indicated above, the task of advancing an endoscope into a tortuous body cavity, such as the colon, is often challenging for a physician. To facilitate the task, the present invention is a control mechanism that aids the determination of the proper orientation of a distal tip of an endoscope for advancement in a desired direction. FIG. 3 shows one technique for controlling the orientation of the distal tip of an endoscope in accordance with the present invention. Although the steps shown in FIG. 3 are described in a particular order, it will be appreciated by those skilled in the art that the steps may be performed in a different order or equivalent steps may be performed to achieve the same results. Therefore, the present invention is not intended to be limited to the particular embodiment described.

Beginning at 100, a processor within the motion control cabinet determines whether a manual control of the distal tip is activated. If so, actuators in the motion control cabinet are activated to tension or release the control wires at 102 as required by the position of a directional switch that is operated by a physician. Processing then returns to 100 to receive the next steering command. If the answer at 100 is no, it is determined at 104 if an automatic control feature is activated. If not, processing returns to 100. Once it has been determined that the automatic control feature has been activated, an image of the tissue is recorded at 106. At 108, a marker indicating the current orientation of the distal tip is made in the image. At 110, the control wires within the endoscope are tensioned as required in order to complete a sweep pattern. At 112, images of the tissue are made at various points in the sweep pattern to image the advancement direction in the body cavity. At 114, the images obtained during the sweep pattern are combined for view by a physician to locate an advancement direction within the body cavity and/or for analysis by a processor. The physician or processor analyzes the combined image to search for a dark spot that typically indicates the direction of desired advancement in the body cavity. However, the physician or processor may analyze the combined image for other information such as the curvature of cavity tissue to determine the advancement direction. At 116, an image processor highlights the determined advancement direction in the body cavity and the steering mechanism activates the control wires to orient the distal tip in the direction of advancement in the cavity.

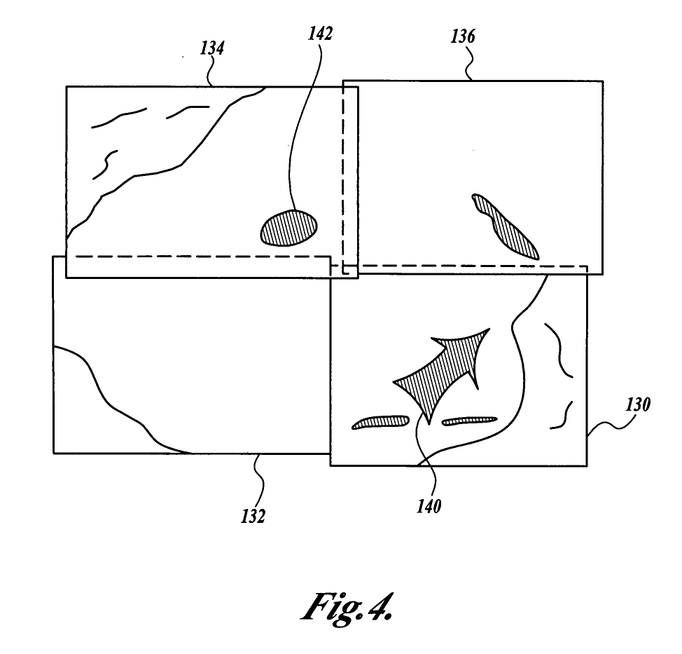

FIG. 4 shows a series of images 130, 132, 134, 136 that may be obtained from a single sweep pattern of the distal tip. Image 130 includes a marker 140 indicating the orientation of the distal tip of the endoscope prior to beginning the sweep pattern. Each of the images 130-136 are aligned and combined by the image processor for display to a physician. The images may be aligned by pattern matching or other image processing techniques to create a larger field of view than is available from any of the individual images. The combined image is then analyzed by the physician or the processor to determine the direction of advancement in the body cavity. In one embodiment, the control cabinet supplies an insufflation gas to inflate the body cavity in the area of the tip of the endoscope prior to performing the sweep pattern. The combined image is then analyzed to find a spot 142 which indicates the direction of advancement within the body cavity. Often this direction is indicated by a relatively dark spot in the image compared to its surroundings. If the physician locates the advancement direction, he or she can highlight it with a mouse or other data input device. The processor within the control cabinet then determines how the control wires should be tensioned in order to orient the distal tip to the direction identified. If the processor determines the advancement direction, the spot 142 may be highlighted or otherwise enhanced for easy viewing by a physician. Once the direction of advancement in the body cavity has been determined, the processor supplies control signals to the steering mechanism in order to orient the distal tip of the endoscope in the desired direction in the body cavity.

With the automatic control feature activated, the physician can reach a desired location in the body simply by pushing or pulling the endoscope and does not have to manually steer the distal tip.

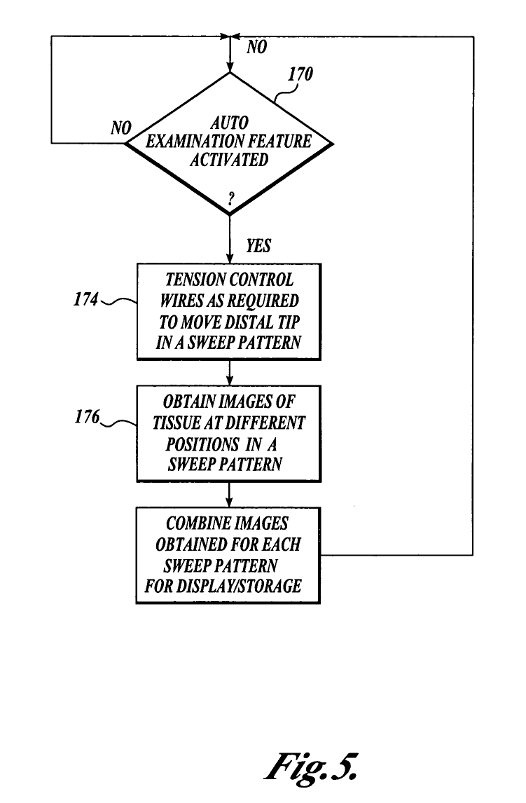

In accordance with another aspect of the present invention, the physician can activate an autoexamination feature that obtains images from a patient's body cavity for view by the physician or storage on a videotape, CD, DVD, etc. As shown in FIG. 5, beginning at 170, a processor determines whether the autoexamination feature has been activated by the physician. If not, the distal tip remains under manual control, and processing returns to 170 until the autoexamination feature has been activated. Once the physician has activated the automatic examination feature, the steering mechanism in the endoscope is controlled to move the distal tip in a repeating sweep pattern. For example, the sweep pattern may be a circular or other pattern such that the distal tip is oriented towards all areas of the body cavity. At 176, images of the tissue in a body cavity are obtained at several positions within the sweep pattern. For example, the autoexamination feature may cause the imaging system to capture four images of the body cavity for each sweep pattern performed. The physician can view each of these images sequentially or the images may be combined to form a larger image for display and analysis by a physician. At any point, the physician can disengage the autoexamination feature and focus the endoscope on a particular site of interest.

As can be seen, the present invention provides a mechanism whereby a physician can perform an endoscopic procedure simply by advancing an endoscope to a point of interest and withdrawing it while the endoscope obtains images of the patient's tissue. The physician is therefore only required to move the endoscope proximally and distally and therefore has more time to focus on the images obtained without having to control the orientation of the distal tip.

While the preferred embodiment of the invention has been illustrated and described, it will be appreciated that various changes can be made therein without departing from the scope of the invention. The scope of the invention is therefore to be determined from the following claims and equivalents thereof.

The embodiments of the invention in which an exclusive property or privilege is claimed are defined as follows:

1. A method of examining a body cavity with a medical device including an elongated shaft that is inserted into the body cavity, an imager for obtaining images of the body cavity, and one or more control wires that are selectively tensioned by one or more actuators in response to signals from a controller that accepts manually input signals to tension the control wires and operates in an autoexamination mode that automatically tensions the control wires to orient the distal tip of the shaft in a repeating sweep pattern, comprising:
inserting the distal tip of the shaft into the body cavity;
advancing the distal tip to a point of interest in the body cavity by manually inputting signals to the controller to orient the distal tip;
activating the autoexamination feature of the medical device upon reaching the point of interest, whereby the actuators are controlled to selectively tension the one or more control wires such that the distal tip is oriented in a repeating sweep pattern until the autoexamination feature is deactivated by a user;
viewing images from the imager as the distal tip is being oriented in the repeating sweep pattern; and
manually withdrawing the shaft as the distal tip is being oriented in the repeating sweep pattern to complete the examination of the body cavity.

2. The method of claim 1, wherein the repeating sweep pattern is circular.

3. The method of claim 1, wherein the repeating sweep pattern directs the distal tip towards all areas of a body cavity.
